(19) United States
(12) Patent Application Publication
(10) Pub. No.: US 2024/0413595 A1
(43) Pub. Date: Dec. 12, 2024

(54) SELF-CORRECTING APPARATUS FOR TRANSMISSION SHAFT AND ULTRASONIC PROBE

(71) Applicant: ULTRASOUND BIOTECHNOLOGY (SHANGHAI) CO., LTD., Shanghai (CN)

(72) Inventors: Weizhong TANG, Shanghai (CN); Minmin ZHOU, Shanghai (CN)

(21) Appl. No.: 18/419,496

(22) Filed: Jan. 22, 2024

Related U.S. Application Data
(63) Continuation of application No. PCT/CN2023/102028, filed on Jun. 25, 2023.

(30) Foreign Application Priority Data
Jun. 8, 2023 (CN) 202310678847.4

(51) Int. Cl. H01R 24/40; A61B 8/00; A61B 8/12; H01R 103/00

(52) U.S. Cl. CPC H01R 24/40; A61B 8/12; A61B 8/445; A61B 8/56; H01R 2103/00; H01R 2201/12

(57) ABSTRACT

Provided herein are a self-correcting apparatus for a transmission shaft and an ultrasonic probe. The self-correcting apparatus includes a rotary inner core, a male connector, a conductive structure which is sleeved outside the male connector and a correcting assembly. A step is arranged inside the rotary inner core. A distal end of the male connector is arranged in the rotary inner core. The correcting assembly is configured for coaxial correction of the male connector and the rotary inner core. The correcting assembly includes an elastic part arranged on the step of the rotary inner core and configured to abut against the distal end of the male connector, and an anti-detaching piece configured to lock the rotary inner core and the male connector. The elastic part is configured to abut against the male connector and to deflect relative to the rotary inner core such that coaxial self-correction of the rotary inner core and the male connector are realized. By arranging the elastic part and the anti-detaching piece between the rotary inner core and the male connector and by matching of the elastic part and the anti-detaching piece, self-correction of the rotary inner core and the male connector can be realized.

### CROSS-REFERENCE TO RELATED APPLICATION

The present application claims priority to Chinese Patent Application No. 2023106788474 filed on Jun. 8, 2023. The contents of the above application are incorporated herein by reference.

### TECHNICAL FIELD

The present invention relates to the technical field of medical equipment, in particular to a self-correcting apparatus for a transmission shaft and an ultrasonic probe.

### BACKGROUND

Bronchial ultrasound, a relatively recent technological advancement, employs an ultrasonic bronchoscope or a micro ultrasonic probe. Using these tools, it navigates through the trachea and bronchial passages, scanning and providing a clear visualization of each layer of the trachea or bronchial wall. This includes displaying adjacent tissue structures around the lumen, such as ultrasonic images of mediastinal lymph nodes. The ultrasonic products used in clinical settings can be broadly categorized into two types. The first type is the ultrasonic bronchoscope, equipped with an ultrasonic probe embedded with an optical fiber structure. It allows scanning in the long axis direction of the airway and can guide real-time needle aspiration biopsies. The second type is the radial bronchial ultrasonic probe, requiring entry into the airway through a bronchoscope's biopsy channel. This probe produces a 360-degree image perpendicular to the airway's axis.

In the radial bronchial ultrasonic probe, a structure of a connecting shaft is mainly used to realize mechanical connection of two rotating shafts. During a connection process of wires, as the wires rotate with the rotating shafts, the two connecting shafts are thus required to realize connection according to a relatively fixed angle to ensure that the wires thereon can be conducted. However, the existing two rotating shafts, during the mechanical connection, cannot realize an effect of self-correction, therefore it is an urgent need for a transmission shaft that can realize self-correction and a rapid connection apparatus for on-shaft signals.

### SUMMARY

The present invention intends to provide a self-correcting apparatus for a transmission shaft and an ultrasonic probe. By arranging an elastic part and an anti-detaching piece between a rotary inner core and a male connector and by matching of the elastic part and the anti-detaching piece, self-correction of the rotary inner core and the male connector can be realized; meanwhile, limited locking of the rotary inner core and the male connector can further be realized; and besides, a combined structure of the elastic part and the anti-detaching piece is simple and reasonable in design, thereby achieving effects of lightness and labor conservation during operation.

In order to achieve the above objectives, the present invention provides a self-correcting apparatus for a transmission shaft, including a rotary inner core, a male connector, a conductive structure sleeved outside the male connector and a correcting assembly, wherein:
a step is arranged inside the rotary inner core;
a distal end of the male connector is arranged in the rotary inner core; and
the correcting assembly is configured for coaxial correction of the male connector and the rotary inner core, the correcting assembly includes an elastic part which is arranged on the step of the rotary inner core and configured to abut against the distal end of the male connector, and an anti-detaching piece configured to lock the rotary inner core and the male connector, and when the rotary inner core is installed into the male connector, the elastic part abuts against the male connector and deflects relative to the rotary inner core such that coaxial self-correction of the rotary inner core and the male connector are realized.

Optionally, the elastic part is a waveform gasket, wherein the waveform gasket is provided with a plurality of convex parts and a plurality of concave parts, the convex parts are alternately connected with the concave parts, the convex parts are configured to abut against the distal end of the male connector, and the concave part is connected with the step of the rotary inner core.

Optionally, the anti-detaching piece includes a movable groove which is arranged on the conductive structure, a pin movably arranged in the movable groove in an axial direction and a pin hole arranged on the rotary inner core and configured to install the pin.

Optionally, a length of a groove cavity of the movable groove in an axial direction is greater than a diameter of the pin, such that the pin can be moveable in the movable groove when the elastic part abuts against the male connector and deflects relative to the rotary inner core.

Optionally, a length of the movable groove in the axial direction is the diameter of the pin plus a length of a gap, and the length of the gap is between zero and a vertical length from a convex tip of the convex part to the step in a natural state.

Optionally, the anti-detaching piece further includes a reset part, the reset part is arranged in the pin hole and wound outside the pin, and two ends of the reset part are respectively fixedly arranged on a side wall of the pin hole and a side wall of the pin.

Optionally, the reset part is a reset spring such that by self-elasticity of the reset spring, the pin is pushed to automatically enter the movable groove.

Optionally, the number of the anti-detaching pieces is N, the N anti-detaching pieces are symmetrically arranged on the rotary inner core with respect to an axial center line of the rotary inner core or annularly arranged on the rotary inner core with equal distances around the axial center line, and N is a positive integer.

Optionally, the conductive structure is made of copper such that electrical connection between the conductive structure and the rotary inner core is realized by connection of the elastic part, and the conductive structure is a ring-shaped or fan-shaped structure.

In order to achieve the above objectives, the present invention further provides an ultrasonic probe, including a female connector, a flexible driving shaft, a transducer, and the self-correcting apparatus for the transmission shaft, wherein: the self-correcting apparatus for the transmission shaft includes the male connector connected with the female connector, the rotary inner core connected with a proximal end of the flexible driving shaft, and the elastic part arranged between the male connector and the step in the rotary inner core. A distal end of the flexible driving shaft is connected with the transducer. The ultrasonic probe further includes a first wire arranged in the flexible driving shaft in a penetrating manner for electrically connecting the transducer with the male connector and the female connector.

Optionally, the male connector includes a first shielding shell, a first anode contact piece and a first cathode contact piece arranged in the first shielding shell, and a first shielding sleeve sleeved outside the first anode contact piece and the first cathode contact piece and configured to fix the first anode contact piece and the first cathode contact piece into the first shielding shell, wherein the first anode contact piece and the first cathode contact piece are respectively connected with an anode and a cathode of the transducer by the first wire.

Optionally, the female connector includes a second shielding shell connected with the first shielding shell, a second anode contact piece and a second cathode contact piece arranged in the second shielding shell, and a second shielding sleeve sleeved outside the second anode contact piece and the second cathode contact piece and configured to fix the second anode contact piece and the second cathode contact piece into the second shielding shell, wherein the first anode contact piece is configured to be in contact with the second anode contact piece, and the first cathode contact piece is configured to be in contact with and electrically connected with the second cathode contact piece.

Optionally, the ultrasonic probe further includes a third shielding sleeve sleeved outside the transducer, and a second wire configured to connect with the third shielding sleeve and the rotary inner core, wherein the third shielding sleeve is electrically connected with the second wire, the rotary inner core, the elastic part, the first shielding shell and the second shielding shell such that conduct electromagnetism on a surface of the transducer can be conducted.

The present invention has the following beneficial effects:
by arranging the elastic part and the anti-detaching piece between the rotary inner core and the male connector and by matching of the elastic part and the anti-detaching piece, self-correction of the rotary inner core and the male connector can be realized; meanwhile, limited locking of the rotary inner core and the male connector can further be realized; and besides, a combined structure of the elastic part and the anti-detaching piece is simple and reasonable in design, thereby achieving effects of lightness and labor conservation during operation.

### BRIEF DESCRIPTION OF THE DRAWINGS

FIG. 1 is a schematic structural diagram of an embodiment in the present invention;

FIG. 2 is a schematic diagram of a three-dimensional structure of the elastic part in FIG. 1 in the present invention;

FIG. 3 is a schematic structural diagram of a second implementation mode of an anti-detaching piece in the present invention;

FIG. 4 is a schematic diagram of a partial structure of an ultrasonic probe in the present invention;

FIG. 5 is a schematic structural diagram of a male connector in the present invention;

FIG. 6 is a schematic structural diagram of a female connector in the present invention.

### REFERENCE NUMBERS IN THE DRAWINGS

1. Rotary inner core; 11. Step;
2. Male connector; 21. Conductive structure; 22. First shielding shell; 23. First anode contact piece; 24. First cathode contact piece; 25. First shielding sleeve;
3. Correcting assembly; 31. Elastic part; 311. Waveform gasket; 3111. Convex part; 3112. Concave part; 32. Anti-detaching piece; 321. Movable groove; 322. Pin; 323. Pin hole; 33. Gap; 34. Reset part;
4. Female connector; 41. Second shielding shell; 42. Second anode contact piece; 43. Second cathode contact piece; 44. Second shielding sleeve;
5. Flexible driving shaft;
6. Transducer; 61. Third shielding sleeve; 62. Insulating tape; 63. Conductive adhesive; 64. Insulating adhesive;
8. Regulating mechanism; 81. Positioning slider; 82. Positioning chute; 83. Inclined platform; 831. The first end; 832. The second end.

### DETAILED DESCRIPTION

In order to make the objectives, technical solutions and advantages of the present invention clearer, the technical solutions in embodiments of the present invention will be clearly and completely described below in conjunction with the accompanying drawings of the present invention. Obviously, the described embodiments are part of, but not all of, the embodiments of the present invention. Based on the embodiments of the present invention, all other embodiments obtained by those of ordinary skill in the art without creative work are within the protection scope of the present invention. Unless otherwise defined, technical or scientific terms used herein should be given their ordinary meanings as understood by those of ordinary skill in the art to which the present invention belongs. As used herein, the terms “comprise” and the like are intended to mean that an element or item appearing before the term encompasses elements or items appearing after the term and their equivalents, but does not exclude other elements or items.

It should be noted that in the present invention, the “distal end” of the apparatus refers to an end that enters a body first, and the “proximal end” refers to an end that enters the body later.

In the ultrasonic probe, a structure of a connecting shaft mainly realizes mechanical connection of two rotating shafts, while in a connection process of wires, as the wires rotate with the rotating shafts, the two connecting shafts are thus required to realize connection according to a relatively fixed angle, in order to ensure that the wires thereon can be conducted. However, the existing two rotating shafts, when in mechanical connection, can not realize an effect of self-correction.

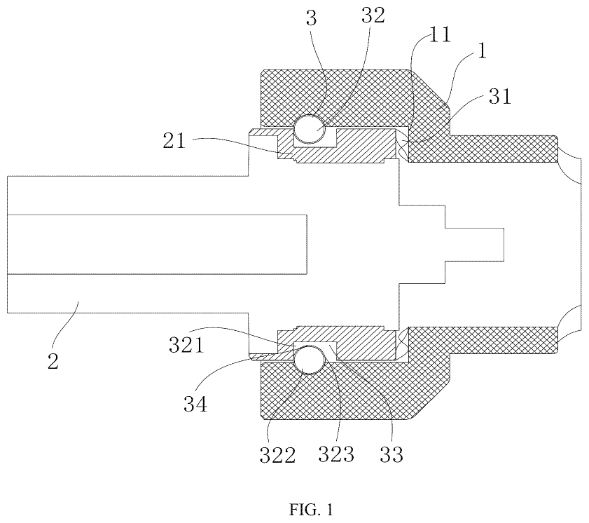

Aiming at the problems existing in the prior art, an embodiment of the present invention provides a self-correcting apparatus for a transmission shaft. FIG. 1 is a schematic structural diagram of an embodiment of the present invention. As shown in FIG. 1, the self-correcting apparatus for the transmission shaft includes a rotary inner core 1 having a step 11 therein, a male connector 2 with a distal end arranged in the rotary inner core 1, a conductive structure 21 which is sleeved outside the male connector 2, and a correcting assembly 3 which is configured for coaxial correction of the male connector 2 and the rotary inner core 1. The correcting assembly 3 includes an elastic part 31 which is arranged on the step 11 of the rotary inner core 1 and abuts against the distal end of the male connector 2, and an anti-detaching piece 32 which is configured to lock the rotary inner core 1 and the male connector 2, and when the rotary inner core 1 is installed into the male connector 2, the elastic part 31 abuts against the male connector 2 and deflects relative to the rotary inner core 1, so as to realize coaxial correction of the rotary inner core 1 and the male connector 2. Specifically, when installation of the rotary inner core 1 and the male connector 2 deflects and coaxiality is failed, the male connector 2 can only be in partial contact with the elastic part 31. At this time, a part of the elastic part 31 in contact with the male connector 2 is compressed, and a part which is not in contact is in a natural telescopic state. In a process of inserting the male connector 2 into the rotary inner core 1 in a horizontal direction, there are two situations as follows: first, the male connector 2 and the rotary inner core 1 are coaxially connected without deflection, and at this time, the distal end of the male connectors 2 is in uniform contact with the elastic part 31; and second, the male connectors 2 is not coaxially connected with the rotary inner core 1, and deflection happens. At this time, part of the male connector 2 is in contact with the elastic part 31, and presses the contacted elastic part 31, and the other part is not in contact therewith. Under elasticity of the elastic part 31, the part of the male connector 2 in contact with the elastic part 31 is pushed to deflect towards the part not in contact, so as to enable the male connector 2 deflects relative to the rotary inner core 1 until the distal end of the male connector 2 is in uniform contact with the elastic part 31 and make force of the part of the elastic part 31 in contact with the male connector 2 uniform. At this time, self-correction of the male connector 2 and the rotary inner core 1 is realized, making a central axis of the male connector 2 and a central axis of the rotary inner core 1 locate on the same horizontal line. In an adjustment process of the embodiment, the self-correction of the rotary inner core 1 and the male connector 2 can be realized in an installation process of the rotary inner core 1 and the male connector 2 without manually operating the self-correction process, thereby reducing operation difficulty of the apparatus and improving assembling efficiency of the apparatus.

By arranging the elastic part 31 and the anti-detaching piece 32 between the rotary inner core 1 and the male connector 2 and by matching of the elastic part 31 and the anti-detaching piece 32, self-correction of the rotary inner core 1 and the male connector 2 can be realized; meanwhile, limited locking of the rotary inner core 1 and the male connector 2 can further be realized; and besides, a combined structure of the elastic part 31 and the anti-detaching piece 32 is simple and reasonable in design, thereby achieving effects of lightness and labor conservation during operation.

When in use, the elastic part 31 is fixed on the step 11 in the rotary inner core 1; the male connector 2 is then inserted into the rotary inner core 1 from left to right and the distal end (which can be understood as a right end) of the male connector 2 is made to abut against the elastic part 31; the anti-detaching piece 32 is then adjusted to lock the male connector 2 and the rotary inner core 1 together, thereby preventing the male connector 2 and the rotary inner core 1 against detachment. In an installation process of the rotary inner core 1 and the male connector 2, under an effect of the elastic part 31, the self-correction of the rotary inner core 1 and the male connector 2 is realized.

It is worth noting that the elastic part 31 can be fixed by adhesive or welding, but is not limited thereto. By adhesive, the adhesive needs to be made of a conductive material, so as to realize electrical conduction between the elastic part 31 and the rotary inner core 1. There is a movable gap in a radial direction between an outer side wall of the conductive structure 21 and an inner side wall of the rotary inner core 1 after installation, so as to make the male connector 2 to deflect in the rotary inner core 1.

In one embodiment, the number of the anti-detaching pieces 32 is N, the N anti-detaching pieces 32 are symmetrically arranged on the rotary inner core 1 with respect to an axial center line of the rotary inner core 1 or annularly arranged on the rotary inner core 1 with equal distances around the axial center line, and N is a positive integer. Due to such arrangement, an effect of better preventing the male connector 2 and the rotary inner core 1 against detachment can be achieved, and meanwhile, a better self-correction effect can further be achieved.

In one embodiment, the conductive structure 21 is made of copper, so as to realize electrical connection between the conductive structure 21 and the rotary inner core 1 by connection of the elastic part 31, and the conductive structure 21 is a ring-shaped or fan-shaped structure. The copper has a property of conducting electricity.

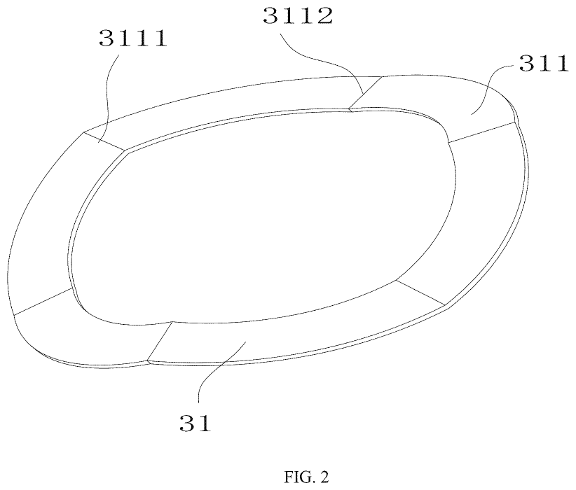

FIG. 2 is a schematic diagram of a three-dimensional structure of the elastic part in FIG. 1 of the present invention. Referring to FIG. 2, the elastic part 31 is a waveform gasket 311, wherein the waveform gasket 311 is provided with a plurality of convex parts 3111 and a plurality of concave parts 3112, the convex parts 3111 are alternately connected with the concave parts 3112, the convex parts 3111 abut against the distal end of the male connector 2, and the concave part 3112 is connected with the step 11 of the rotary inner core 1. The convex part 3111 has a certain elasticity, so the elastic part 31 in this embodiment can realize the self-correction of the rotary inner core 1 and the male connector 2. When in use after correction, the elasticity of the elastic part 31 can play a role in absorbing vibration, thereby protecting the apparatus.

In one embodiment, a first implementation mode of the anti-detaching piece 32 is provided. Specifically referring to FIG. 1, the anti-detaching piece 32 includes a movable groove 321 which is arranged on the conductive structure 21, a pin 322 which is movably arranged in the movable groove 321 in an axial direction and a pin hole 323 which is arranged on the rotary inner core 1 and configured to install the pin 322. On one hand, the arrangement of the movable groove 321 and the pin hole 323 can play a role of positioning, specifically, installation positions of the male connector 2 and the rotary inner core 1 can be quickly located by the movable groove 321 and the pin hole 323. On the other hand, after the male connector 2 is installed in the rotary inner core 1, the pin 322 penetrates through the pin hole 323 to be inserted into the movable groove 321, so as to lock the male connector 2 and the rotary inner core 1 and prevent the male connector 2 and the rotary inner core 1 against detachment.

In one embodiment, a length of a groove cavity of the movable groove 321 in an axial direction is greater than a diameter of the pin 322, such that the pin 322 can move in the movable groove 321 when the elastic part 31 abuts against the male connector 2 and deflects relative to the rotary inner core 1. In one embodiment, a height of the groove cavity of the movable groove 321 in the axial direction is greater than the diameter of the pin 322, such that the pin 322 can be moveable within the movable groove 321 when the elastic part 31 abuts against the male connector 2 and deflects relative to the rotary inner core 1. It can be seen that the pin 322 is suspended in the movable groove 321 and is not in contact with the groove wall of the movable groove 321. Therefore, when the elastic part 31 drives the male connector 2 and the rotary inner core 1 to realize self-correction, the pin 322 does not hinder the self-correction process. The male connector 2 can thus not only move axially relative to the rotary inner core 1 (which can be understood as moving left and right in a horizontal direction), but also move radially relative to the rotary inner core 1 (which can be understood as moving up and down in a vertical direction). In this way, the self-correction process of the male connector 2 and the rotary inner core 1 can be better realized.

In one embodiment, the length of the movable groove 321 in the axial direction is the diameter of the pin 322 plus a length of a gap 33 (the length of the gap 33 can be understood to be the length of the groove cavity of the movable groove 321 in the axial direction minus the length beyond the diameter of the pin 322), the length of the gap 33 is between zero and a vertical length from a convex tip of the convex part 3111 to the step 11 in a natural state. Due to such arrangement, after the male connector 2 and the rotary inner core 1 are self-corrected, the male connector 2 can not only be in contact with the elastic part 31, the elastic part 31 is compressed, the pin 322 can also abut against the right-side wall of the movable groove 321 under abutting of the elastic part 31. In this way, at least one side of the pin 322 can be ensured to be in contact with the wall of the movable groove 321, and the abutting between the pin 322 and the side wall of the movable groove 321 can make the positions of the male connector 2 and the rotary inner core 1 more stable after self-correction.

In one embodiment, the anti-detaching piece 32 further includes a reset part 34, the reset part 34 is arranged in the pin hole 323 and wound outside the pin 322, and two ends of the reset part 34 are respectively fixedly arranged on a side wall of the pin hole 323 and a side wall of the pin 322, as shown in FIG. 1. The reset part 34 is configured to drive the pin 322 to automatically enter the movable groove 321 without manual control, making the installation process of the apparatus more intelligent and more convenient. In use, when the male connector 2 is inserted rightward into the rotary inner core 1, the male connector 2 will move outward the rotary inner core 1 by the conductive structure 21 abutting against the pin 322. When the movable groove 321 is aligned with the pin hole 323, the pin 322 is pushed by a reset force of the reset part 34 and will move into the rotary inner core 1 and enter the movable groove 321, so as to lock the rotary inner core 1 and the male connector 2 and prevent the rotary inner core 1 and the male connector 2 against detachment.

In one example, the reset part 34 is preferably a reset spring, such that by self-elasticity of the reset spring the pin 322 is pushed to automatically enter the movable groove 321. Of course, the reset part is not limited to the reset spring.

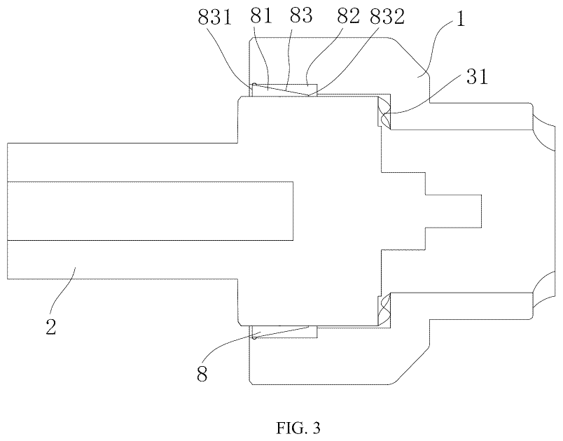

In one embodiment, a second implementation mode of the anti-detaching piece 32 is further provided. FIG. 3 is a schematic structural diagram of the second implementation mode of the anti-detaching piece in the present invention. Referring to FIG. 3, the anti-detaching piece 32 includes a positioning slider 81 which is arranged on the outer side wall of the male connector 2 or the inner side wall of the rotary inner core 1, and a positioning chute 82 which is arranged on the outer side wall of the male connector 2 or the inner side wall of the rotary inner core 1, and the positioning slider 81 is arranged in the positioning chute 82 in a sliding manner. In this embodiment, the arrangement of the combined structure of the positioning slider 81 and the positioning chute 82 can achieve the effect of accurate positioning. Specifically, by the combined structure of the positioning slider 81 and the positioning chute 82, the installation positions of the rotary inner core 1 and the male connector 2 can be located accurately. It can be seen that the second implementation mode of the anti-detaching piece 32 can achieve a more accurate positioning effect than the first implementation mode, making the installation process of the rotary inner core 1 and the male connector 2 more convenient, time-saving and labor-saving.

In one example, an inclined platform 83 is arranged on one side, far away from the outer side wall of the male connector 2 or the inner side wall of the rotary inner core 1, of the positioning slider 81, the inclined platform 83 is provided with a first end 831 and a second end 832, and a distance from the first end 831 to an axial center line of the male connector 2 is greater than that from the second end 832 to the axial center line. Specifically, in the example of FIG. 3, the inclined platform 83 is high on a left and low on a left. Due to such arrangement, on one hand, the positioning slider 81 can better move rightwards in the positioning chute 82. On the other hand, during the rightward movement of the positioning slider 81, as the distance from the inclined platform 83 to the axial center line becomes larger and larger, when the male connector 2 and the rotary inner core 1 are installed obliquely, under guidance of an inclined surface of the inclined platform 83, by abutting against a bottom wall of the positioning chute 82, the proximal end of the male connector 2 will rotate counterclockwise toward the axial center line of the male connector 2, so as to achieve the effect of self-correction. In this embodiment, the inclined platform 83 can play a role of first-step self-correction, and the elastic part 31 is arranged to assist the inclined platform 83 for self-correction, that is, to cooperate with the inclined platform 83 to make the male connector 2 and the rotary inner core 1 achieve better self-correction. The elastic part 31 is arranged further for absorbing vibration, and by vibration absorption, the apparatus can be protected. It can be seen that the second implementation mode of the anti-detaching piece 32 can achieve a better correction effect in case of self-correction than the first implementation mode. Meanwhile, in the second embodiment, the self-correction process is finished during the installation of the rotary inner core 1 and the male connector 2. Of course, in some implementation modes, the inclined platform 83 is configured to replace the elastic part 31 to play a role of self-correction.

In another example, a first protrusion protrudes at the first end 831 of the inclined platform 83 in a direction far away from the central axis of the male connector 2, or a first groove is recessed in a direction close to the central axis of the male connector 2. A second groove is recessed at one side, close to the first end 831 of the inclined platform 83, of the bottom wall of the positioning chute 82 in a direction far away from the central axis of the male connector 2, or a second protrusion protrudes in a direction close to the central axis of the male connector 2. The first protrusion is clamped and matched with the second groove, the first groove is clamped and matched with the second protrusion, and by the matching of the first protrusion and the second groove or the first groove and the second protrusion, limited locking of the male connector 2 and the rotary inner core 1 can be realized.

The self-correcting apparatus for the transmission shaft in the present invention is used in the field of ultrasonic probes, but is not limited to the field of ultrasonic probes.

In electronic equipment, electromagnetic interference energy is generally transmitted by conductive coupling and radiative coupling. In order to meet requirements of electromagnetic compatibility, a filter technology needs to be adopted for conductive coupling, that is, an EMI filter apparatus is adopted for suppression; and for radiative coupling, a shielding technology is adopted for suppression. The importance thereof is more prominent as equipment and system electromagnetic environments are deteriorating caused by factors such as increasing density of the current electromagnetic spectrum, dramatical increasing of the electromagnetic power density per unit volume, massive mixed use of high and low level apparatuses or equipment and the like.

Shielding is a method of confining electromagnetic waves to a certain area through shielding bodies such as a shell, a box and a plate made of metal. As radiation sources are divided into an electric field source and a magnetic field source in a proximal area and plane waves in a distal area, shielding performance of a shielding body is different according to a different radiation source in aspects such as material selection, a structure shape and hole leakage control. In order to achieve the required shielding performance in the design, it is necessary to first determine a radiation source and a frequency range, and then determine control factors according to a typical leakage structure of each frequency band, and then select an appropriate shielding material and design a shielding shell.

The two are separated in the prior art, one is an anode and the other is cathode. A male anode is connected with a female anode and a male cathode is connected with a female cathode, which is vulnerable to external signal interference. Specifically, an image at a transducer end is vulnerable to external interference, which brings trouble to operator's diagnosis and easily leads to misdiagnosis. In the prior art, external interference is connected to a host for software processing. However, as the software processing cannot perform safe and effective judgment on various conditions based on clinic practice, misdiagnosis still exists.

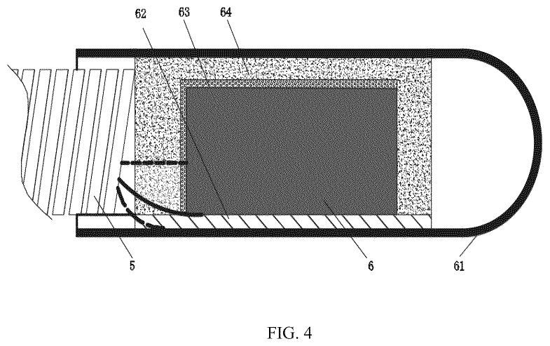

Aiming at the problems existing in the prior art, an embodiment of the present invention also provides an ultrasonic probe. FIG. 4 is a schematic diagram of a partial structure of an ultrasonic probe in the present invention. Referring to FIG. 4, the ultrasonic probe includes a female connector 4, a flexible driving shaft 5, a transducer 6, and the self-correcting apparatus for the transmission shaft. The self-correcting apparatus for the transmission shaft includes the male connector 2 which is connected with the female connector 4, the rotary inner core 1 which is connected with a proximal end of the flexible driving shaft 5, and the elastic part 31 which is arranged between the male connector 2 and the step 11 in the rotary inner core 1, and a distal end of the flexible driving shaft 5 is connected with the transducer 6. The ultrasonic probe further includes a first wire which is arranged in the flexible driving shaft 5 in a penetrating manner for electrically connecting the transducer 6 with the male connector 2 and the female connector 4. Preferably, the rotary inner core 1 is made of stainless steel, but is not limited to stainless steel.

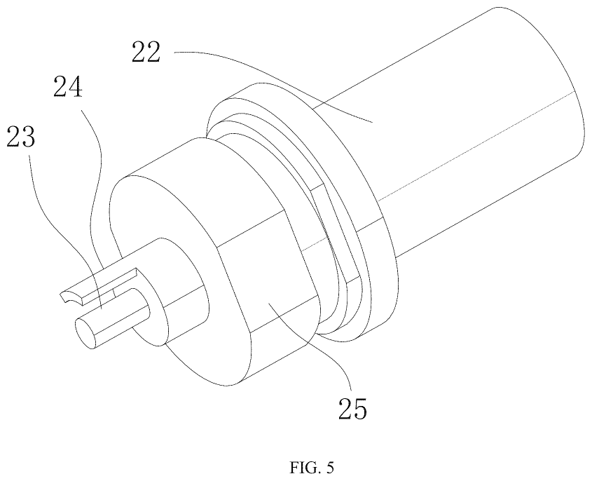

FIG. 5 is a schematic structural diagram of the male connector 2 in the present invention. Referring to FIG. 5, the male connector 2 includes a first shielding shell 22, a first anode contact piece 23 and a first cathode contact piece 24 which are arranged in the first shielding shell 22, and a first shielding sleeve 25 which is sleeved outside the first anode contact piece 23 and the first cathode contact piece 24 and fixes the first anode contact piece 23 and the first cathode contact piece 24 in the first shielding shell 22, and the first anode contact piece 23 and the first cathode contact piece 24 are respectively connected with an anode and a cathode of the transducer 6 by the first wire. Preferably, the first shielding shell 22 is made of stainless steel, but is not limited to stainless steel. The first shielding sleeve 25 is made of rubber, but is not limited to rubber. In this embodiment, the first shielding shell 22 is sleeved outside the first anode contact piece 23 and the first cathode contact piece 24, so as to form a one-side metal shielding layer outside the first anode contact piece 23 and the first cathode contact piece 24 to shield external interference signals.

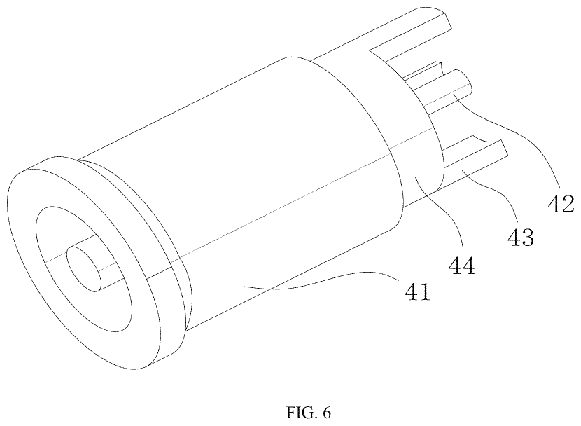

FIG. 6 is a schematic structural diagram of the female connector 4 in the present invention. Referring to FIG. 6, the female connector 4 includes a second shielding shell 41 which is connected with the first shielding shell 22, a second anode contact piece 42 and a second cathode contact piece 43 which are arranged in the second shielding shell 41, and a second shielding sleeve 44 which is sleeved outside the second anode contact piece 42 and the second cathode contact piece 43 and fixes the second anode contact piece 42 and the second cathode contact piece 43 in the second shielding shell 41, the first anode contact piece 23 is in contact with the second anode contact piece 42, and the first cathode contact piece 24 is in contact with and electrically connected with the second cathode contact piece 43. Preferably, the second shielding shell 41 is made of stainless steel, but is not limited to stainless steel. The second shielding sleeve 44 is made of rubber, but is not limited to rubber. In this embodiment, the second shielding shell 41 is sleeved outside the second anode contact piece 42 and the second cathode contact piece 43, so as to form a one-side metal shielding layer outside the second anode contact piece 42 and the second cathode contact piece 43 to shield external interference signals.

In one embodiment, the ultrasonic probe further includes a third shielding sleeve 61 which is sleeved outside the transducer 6, and a second wire which is configured to connect the third shielding sleeve 61 and the rotary inner core 1, wherein the third shielding sleeve 61, the second wire, the rotary inner core 1, the elastic part 31, the first shielding shell 22 and the second shielding shell 41 are electrically connected, so as to conduct electromagnetism on a surface of the transducer 6. The third shielding sleeve 61 is made of stainless steel, but is not limited to stainless steel.

When in use, work of the transducer 6 will generate electromagnetism on the surface, thus forming noise interference, and further affecting a test result. The present invention improves the structure of the existing ultrasonic probe, and the improved ultrasonic probe can conduct the electromagnetism generated by the transducer 6, thereby preventing the noise interference from affecting the monitoring result of the ultrasonic probe. Specifically, in the present invention, a first series circuit has the following connection relationship in sequence: the first anode contact piece 23, the second anode contact piece 42, the first wire, the cathode of the transducer 6, the anode of the transducer 6, the first wire, the first cathode contact piece 24, the second cathode contact piece 43, and an anode and a cathode of a power supply. The series circuit is used for controlling the work of the transducer 6, to monitor a target area. In the present invention, a second circuit has the following connection relationship in sequence: the third shielding sleeve 61, the second wire, the rotary inner core 1, the elastic part 31, the first shielding shell 22 and the second shielding shell 41 are electrically connected, and the second shielding shell 41 is electrically connected with a ground wire (not limited to the ground wire) to conduct electromagnetic signals generated on the surface of the transducer 6, thereby eliminating the noise interference caused by the electromagnetic signals and further making the monitoring result more accurate. Specifically, in the prior art, the anode contact piece penetrates into the cathode contact piece, and no shielding layer is arranged outside the two, thereby being vulnerable to external signal interference. However, in the present invention, the rotary inner core 1, the first shielding shell 22 and the second shielding shell 41 are combined to form a shielding structure outside the first anode contact piece 23, the second anode contact piece 42, the first cathode contact piece 24 and the second cathode contact piece 43, thereby forming a shielding layer outside the anode contact piece and the cathode contact piece and effectively preventing external signal interference.

In one embodiment, the ultrasonic probe further includes an insulating tape 62 which is arranged between the transducer 6 and the third shielding sleeve 61, a conductive adhesive 63 which is sleeved outside the transducer 6, and an insulating adhesive 64 which is sleeved outside the conductive adhesive 63 and the transducer 6. The insulating adhesive 64 is connected with the insulating tape 62, and a closed cavity is formed between the two. The transducer 6 is arranged in the closed cavity to form a fully closed insulation structure outside the transducer 6, as shown in FIG. 4. In the present invention, the transducer 6 has a better insulation performance due to the combined structure of the insulating tape 62 and the insulating adhesive 64.

It is worth noting that in the present invention, each connection position needs to be treated by dispensing and sealing, which will not be described here.

While the implementation modes of the present invention have been described in detail above, it will be apparent to those skilled in the art that various modifications and variations can be made to the implementation modes. It is to be understood, however, that such modifications and variations are within the scope and spirit of the present invention as set forth in the claims. Moreover, the present invention described herein may have other implementation modes and may be implemented or realized in various ways.

1. A self-correcting apparatus for a transmission shaft, comprising a rotary inner core, a male connector, a conductive structure sleeved outside the male connector and a correcting assembly, wherein:
a step is arranged inside the rotary inner core;
a distal end of the male connector is arranged in the rotary inner core; and
the correcting assembly is configured for coaxial correction of the male connector and the rotary inner core, the correcting assembly comprises an elastic part arranged on the step of the rotary inner core and configured to abut against the distal end of the male connector, and an anti-detaching piece configured to lock the rotary inner core and the male connector, and when the rotary inner core installed into the male connector, the elastic part abuts against the male connector and deflects relative to the rotary inner core such that coaxial self-correction of the rotary inner core and the male connector are realized.

2. The self-correcting apparatus for the transmission shaft of claim 1, wherein the elastic part is a waveform gasket, the waveform gasket is provided with a plurality of convex parts and a plurality of concave parts, the convex parts are alternately connected with the concave parts, the convex parts are configured to abut against the distal end of the male connector, and the concave part is connected with the step of the rotary inner core.

3. The self-correcting apparatus for the transmission shaft of claim 2, wherein the anti-detaching piece comprises a movable groove arranged on the conductive structure, a pin movably arranged in the movable groove in an axial direction, and a pin hole arranged on the rotary inner core and configured to install the pin.

4. The self-correcting apparatus for the transmission shaft of claim 3, wherein a length of a groove cavity of the movable groove in an axial direction is greater than a diameter of the pin such that the pin can be moveable within the movable groove when the elastic part abuts against the male connector and deflects relative to the rotary inner core.

5. The self-correcting apparatus for the transmission shaft of claim 3, wherein the length of the groove cavity of the movable groove in the axial direction is the diameter of the pin plus a length of a gap, the length of the gap being between zero and a vertical length from a convex tip of the convex part to the step in a natural state.

6. The self-correcting apparatus for the transmission shaft of claim 3, wherein the anti-detaching piece further comprises a reset part, the reset part being arranged in the pin hole and wound outside the pin, and two ends of the reset part being respectively fixedly arranged on a side wall of the pin hole and a side wall of the pin.

7. The self-correcting apparatus for the transmission shaft of claim 6, wherein the reset part is a reset spring such that by self-elasticity of the reset spring the pin is pushed to automatically enter the movable groove.

8. The self-correcting apparatus for the transmission shaft of claim 1, wherein the number of the anti-detaching pieces is N, the N anti-detaching pieces being symmetrically arranged on the rotary inner core with respect to an axial center line of the rotary inner core or annularly arranged on the rotary inner core with equal distances around the axial center line, and N being a positive integer.

9. The self-correcting apparatus for the transmission shaft of claim 1, wherein the conductive structure is made of copper such that electrical connection between the conductive structure and the rotary inner core is realized by connection of the elastic part, the conductive structure being a ring-shaped or fan-shaped structure.

10. An ultrasonic probe, comprising a female connector, a flexible driving shaft, a transducer, and the self-correcting apparatus for the transmission shaft of claim 1, wherein:
the self-correcting apparatus for the transmission shaft comprises the male connector connected with the female connector, the rotary inner core connected with a proximal end of the flexible driving shaft, and the elastic part arranged between the male connector and the step in the rotary inner core, a distal end of the flexible driving shaft being connected with the transducer, and the ultrasonic probe further comprising a first wire arranged in the flexible driving shaft in a penetrating manner for electrically connecting the transducer with the male connector and the female connector.

11. The ultrasonic probe of claim 10, wherein the male connector comprises a first shielding shell, a first anode contact piece and a first cathode contact piece arranged in the first shielding shell, and a first shielding sleeve sleeved outside the first anode contact piece and the first cathode contact piece and configured to fix the first anode contact piece and the first cathode contact piece into the first shielding shell, the first anode contact piece and the first cathode contact piece being respectively connected with an anode and a cathode of the transducer by the first wire.

12. The ultrasonic probe of claim 11, wherein the female connector comprises a second shielding shell connected with the first shielding shell, a second anode contact piece and a second cathode contact piece arranged in the second shielding shell, and a second shielding sleeve sleeved outside the second anode contact piece and the second cathode contact piece and configured to fix the second anode contact piece and the second cathode contact piece into the second shielding shell, the first anode contact piece configured to be in contact with the second anode contact piece, and the first cathode contact piece configured to be in contact with and electrically connected with the second cathode contact piece.

13. The ultrasonic probe of claim 12, further comprising a third shielding sleeve sleeved outside the transducer, and a second wire configured to connect with the third shielding sleeve and the rotary inner core, wherein the third shielding sleeve is electrically connected with the second wire, the rotary inner core, the elastic part, the first shielding shell and the second shielding shell such that electromagnetism on a surface of the transducer can be conducted.
